(19) United States
(12) Patent Application Publication
(10) Pub. No.: US 2002/0012641 A1
(43) Pub. Date: Jan. 31, 2002

(54) Use of EGF-R protein tyrosine kinase inhibitors for preventing photoaging in human skin

(76) Inventors: John J. Voorhees, Ann Arbor, MI (US); Gary J. Fisher, Ypsilanti, MI (US)

Correspondence Address: Hopgood, Calimafde, Kalil & Judlowe, LLP, Suite 4000, 60 East 42nd St., New York, NY, 10165, US

(21) Appl. No.: 09/891,881

(22) Filed: Jun. 26, 2001

Related U.S. Application Data
(60) Provisional application No. 60/213,940, filed on Jun. 26, 2000.

(51) Int. Cl. A61K 7/42; A61K 31/519

(52) U.S. Cl. 424/59; 514/456; 514/258; 514/725

(57) ABSTRACT

Photoaging of human skin, such as evidenced by the increased presence of matrix metalloproteinases after exposure to UV radiation, is prevented by pretreating the skin with an inhibitor of epidermal growth factor receptor (EGF-R) prior to exposure. Such inhibitor are preferably natural, an example of which is genistein. Compositions used for such purposes preferably include an EGF-R as well as another MMP inhibitor, such as a retinoid.

### RELATED APPLICATIONS

This application is based on provisional application No. 60/213940, filed Jun. 26, 2000, the disclosure and figures of which are incorporated herein in their entirety.

### BACKGROUND OF THE INVENTION

1. Field of the Invention

This invention relates to new methods for using tyrosine kinase inhibitors, more specifically epidermal growth factor receptor (EGF-R) inhibitors, in the prevention and treatment of photoaging in human skin, especially photoaging from ultraviolet radiation, and most especially from the sun.

2. The State of the Art

Our prior patents, U.S. Pat. Nos. 5,837,224 and 6,130,254 (the disclosures of which are incorporated herein by reference), describe photoaging in human skin by UV radiation, especially from the sun. As described therein, UV radiation causes, among other effects, an increase in enzymes that degrade collagen; one class of such enzyme is called a matrix metalloproteinase, abbreviated as MMP. The existence of MMPs in skin is caused by what is believed to be UV-initiated signalling along both the stress-activated pathway (SAP) and the mitogen-activated pathways (MAP). These pathways activate the transcription factor AP-1, which results in increased MMP production in UV-exposed skin. Our prior patents teach that application of a retinoid to human skin prior to UV exposure reduces subsequent MMP-mediated collagen degradation.

Our co-pending application Ser. No. 28,435, filed Feb. 28, 1998, describes choronological aging in human skin. Skin that is essentially sun-protected during life (e.g., skin on the hip or buttock area) nevertheless shows some of the same etiology as skin that is effected by typical UV radiation exposure (e.g., skin on the face and forearms); namely, down-regulated collagen synthesis and upregulated MMP activity. In elderly skin, levels of AP-1 are upregulated almost as if the sun-protected skin had been exposed to UV radiation on a daily basis. Our co-pending application teaches that application of a retinoid to sun-protected human skin normalizes the skin by reducing MMP levels and by increasing collagen synthesis.

Our co-pending application No. 285,860, filed Apr. 2, 1999, describes the reduction in collagen biosynthesis in human brought about by UV-irradiation. As described therein, UV irradiation of human skin not only induces enzymes (MMPs) that degrade collagen in the dermal matrix, it also inhibits the biosynthesis of collagen. Thus, UV irradiation not only causes degradation of the collagen structure, it also prevents its reconstruction.

Protein tyrosine kinases are involved in regulating critical functions in mammalian cells (e.g., cell growth, cell death, inflammation, and so on). There are two classes of protein tyrosine kinases: receptor protein tyrosine kinases and nonreceptor protein tyrosine kinases. Many growth factor receptors on cell surfaces have intrinsic protein tyrosine kinase activity, so that when the growth factor binds to the cognate receptor on the cell surface it stimulates the intracellular protein tyrosine kinase activity. This intrinsic activation initiates a signal transduction cascade that typically results in cell growth and survival (e.g., effects expected from growth factors).

In the field of cancer research, much work has been done on protein tyrosine kinase inhibitors. The inhibitor compounds inhibit the activity of protein kinases such as tyrosine kinases and may act reversibly or irreversibly on the kinase to prevent its activation. The epidermal growth factor receptor family (e.g., EGF-R or ErbB), the platelet-derived growth factor receptor family (PDGF), and the fibroblast growth factor receptor family (FGF-R) possess intrinsic tyrosine kinase activity. These growth factor receptors are believed to be important in cancer pathophysiology because when they are mis-regulated they lead to uncontrolled cell proliferation, leading to tumor growth and/or metastases. Some of the following patents disclose inhibitors stated therein as useful for skin hyperproliferative diseases such as psoriasis. Various patents disclosing protein tyrosine kinase inhibitors include the following U.S. Pat Nos. 5,840,883; 5,935,993; 5,891,917; 5,773,459; 5,710,173; 5,686,457; 5,656,655; 5,650,415; 5,929,081; 5,760,041; 5,886,020; 5,880,141; 5,880,130; 5,869,485; 5,840,880; 5,834,504; 5,763,470; 5,374,652; 5,302,606; 5,108,921; 5,196,446; 5,914,343; and 5,911,995; the disclosures of which are incorporated herein by reference. Other protein tyrosine kinase inhibitors are described in the following abstracts: T. Ohmori et al., “Cellular stresses can modulate the sensitivity of human carcinoma cells to EFGR kinase inhibitors,” Proc.

Amer. Assoc. Cancer Res., 40, March 1999; H. Mett et al., “CGP 59326, a potent protein tyrosine kinase (PTK) inhibitor which selectively blocks growth of epidermal growth factor receptor (EGFR) expressing tumor cells,” Proc. Amer. Assoc. Cancer Res., 39, March 1998; E. Suárez et al., “Dephosphorylation of the epidermal growth factor is modulated by ganglioside GM3,” Proc. Amer. Assoc. Cancer Res., 39, March 1998; G. J. Kelloff et al., “Epidermal Growth Factor Receptor Tyrosine Kinase Inhibitors as Potential Cancer Chemopreventives,” Cancer Epidemiology, Biomarkers & Prevention, 5, 657-666, August 1996; J. D. Moyer et al., “Induction of Apoptosis and Cell Cycle Arrest by CP-358,774, an Inhibitor of Epidermal Growth Factor Receptor Tyrosine Kinase,” Cancer Res., 57, 4838-4848, Nov. 1, 1997; M. N. Lango et al., “Modulation of TGF-α/EGFR autocrine signaling by a novel RAR-selective retinoid (LGD 1550),” Proc. Amer. Assoc. Cancer Res., 40, March 1999; D. W. Fry et al. “Specific, irreversible inhibitors of the epidermal growth factor receptor (EGFR) family of tyrosine kinases,” Proc. Amer. Assoc. Cancer Res., 39, March 1998; J. M. Nelson et al., “In vitro comparison of irreversible versus reversible inhibition for a series of substituted quinazolines and pyridopyrimidines that are potent and specific inhibitors of the epidermal growth factor receptor (EGFR) family of tyrosine kinases,” Proc. Amer. Assoc. Cancer Res., 39, March 1998; A. J. Kraker et al., “In vivo antitumor activity of selective c-src tyrosine kinase (TK) inhibitors,” Proc. Amer. Assoc. Cancer Res., 40, March 1999; S. Cockerill et al., “The design of indazolylaminoquinazolines and pyridopyrimidines as inhibitors of class-1 receptor tyrpsine kinases,” Proc. Amer. Assoc. Cancer Res., 40, March 1999; P. W. Vincent, “Characterization of the in vivo activity of a novel EGF receptor family kninase inhibitor, PD 169414,” Proc. Amer. Assoc. Cancer Res., 39, March 1998; S. J. Patmore et al., “In vivo evaluation of the irreversible EGF receptor tyrosine kinase inhibitor PD 168393,” Proc. Amer. Assoc. Cancer Res., 39, March 1998; and L. J. McCawley et al., “Receptor tyrosine kinases require sustained activiation of MAPK and NJK/SAPK for migration and induction of 92 kDa gelatinase,” Proc. Amer. Assoc. Cancer Res., 39, March 1998 (regarding MEK-1 inhibitor PD 98059); the disclosures of which are incorporated herein by reference.

Various EGF-R inhibitors including AG-494 (a member of the tyrphostin family of tyrosine kinase inhibitors), AG-825 (5-[(Benzthiazol-2-yl)thiomethyl]-4-hydroxy-3-methoxybenzylidenecyanoacetamide), AG-1478 (4-(3-Chloroanilino)-6,7-dimethoxyquinazoline), EI-146 (an Erbstatin analog), Methyl 2,5-dihydroxycinnamate, HDBA (2-Hydroxy-5-(2,5-dihydroxybenzylamino)-2-hydroxybenzoic acid; Onoda et al., J. Natural Products, 52:1252, 1989), Lavendustin A, RG-13022 (a non-phenolic tyrphostin analog which inhibits the EGF receptor), RG-14620 (a non-phenolic tyrphostin analog which is selective for the EGF receptor and long acting), Tyrphostin 23 (RG-50810), Tyrphostin 25 ([(3,4,5-trihydroxyphenyl)-methylene]-propanedinitrile, Gazit et al., J. Med. Chem., 32:2344, 1989; also known as RG-50875), Tyrphostin 46, Tyrphostin 47 (RG-50864, AG-213), Tyrphostin 51, and Tyrphostin 1. Certain inhibitors of protein tyrosine kinase are specific inhibitors at lower concentrations, yet may inhibit other protein tyrosine kinases at higher concentrations.

A review article by S. B. Noonberg and C. C. Benz (“Tyrosine Kinase inhibitors Targeted to the Epidermal Growth Factor Receptor Subfamily—Role as Anticancer Agents”, Drugs, 2000 Apr:59(4)) (the disclosure of which is incorporated herein by reference) describes various approaches for inhibiting the kinase activity of EGF receptors, including antibodies, immunotoxin conjugates, ligand-binding cytotoxic agents, and small molecule kinase inhibitors.

### SUMMARY OF THE INVENTION

In light of the foregoing, it would be beneficial to identify additional compounds that inhibit UV-inducement of MMPs in human skin. It would be especially beneficial to identify such compounds that can be adminstered topically.

Thus, in one aspect this invention provides a method for inhibiting photoaging of human skin by application to the skin, prior to UV exposure, of an inhibitor of EGF-R. Natural compounds, such as genistein (a soy isoflavone), are preferred.

In another aspect, this invention provides a composition for inhbiting photoaging of human skin, which comprises a combination of UVA and UVB blockers, as well as an EGF-R inhibitor, and preferably an additional MMP inhibitor such as a retinoid, a direct acting MMP inhibitor (such as Galardin), and/or a compound that inhibits the cytochrome P-450 mediated degradation of retinoids.

### BRIEF DESCRIPTION OF THE DRAWINGS

FIG. 1 is a cartoon showing two pathways by which UV radiation from the sun may cause photoaging in human skin.

FIGS. 2-5 are the results of in vivo testing of human subjects' skin exposed to UV radiation and then biopsied, wherein their skin had been pretreated with a genistein solution to determine the effect on the expected increase in, respectively, JNK activation, cJUN protein, MMP-1 mRNA, and EGF-R phosphorylation after exposure of the skin to UV radiation.

### DESCRIPTION OF THE INVENTION

This invention provides compositions and methods for inhibiting MMP formation; the compositions and methods are believed to work by inhibiting the growth factor receptor pathways responsible for these detrimental effects in UV-irradiated human skin.

We have found that UV radiation activates, among other pathways, the epidermal growth factor (EGF) receptor protein tyrosine kinase (PTK) in human skin. The receptor for EGF, EGF-R, is also known as ErbB, and is part of the ErbB family of receptors. Activation of the EGF-R causes activation of its intrinsic PTK activity and leads to MMP upregulation.

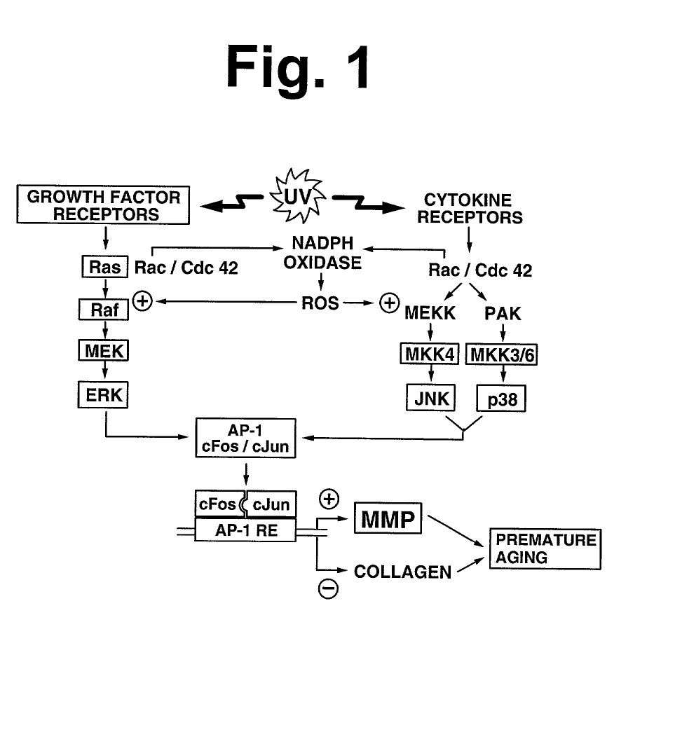

While not desirous of being constrained to a particular theory of operation, we believe we have discovered that multiple receptor-mediated pathways are activated by UV irradiation in human skin and that lead to increased MMPs are dependent predominantly upon EGF-R activation. That is, EGF-R activation by UV preceeds and is required for activation of other pathways that lead to MMP induction in human skin. Thus, by blocking UV activation of EGF-R with the use of specific EGF-R PTK inhibitors, one can block UV induction of MMPs. In essence, we have discovered that administration of PTK inhibitors of EGF-R prevent UV-induced photoaging (by collagen degradation) in human skin. As shown in the cartoon of FIG. 1, UV radiation from the sun activates both cytokine receptors and growth factor receptors. Each receptor, though its own signalling pathway, results in the creation of activated protein-1 (AP-1), a heterodimer of cJUN and cFOS proteins. In human skin, the concentration of cFOS remains essentially constant (see G. J. Fisher and J. J. Voorhees, “Molecular Mechanisms of Photoaging and its Prevention by Retinoic Acid,” JID Symposium Proc., vol. 3, no. 1, pp. 61-68 (Aug. 1998)); it is the concentration of cJUN that varies as does UV exposure of the skin. The AP-1 receptor element (RE) is activated thereby, and causes the increase in MMPs and a concomitant decrease in collagen biosynthesis. The ROS (reactive oxygen species) present in human skin (e.g., induced by solar radiation) activate both pathways. This invention primarly concerns inhibiting the growth factor receptor pathway by which EGF-R functions, although it should be apparent from FIG. 1 that inhibiting both of the receptor pathways would be beneficial for inhibiting photoaging of human skin. In fact, our results indicate that all direct EGF-R inhibitors actually inhibit both of these pathways.

To determine which factors are required for signalling particular to induction of MMPs in UV-irradiated human skin, or further signalling leading to MMP formation, various testing was done.

Experiments

As noted above, the EGF-R molecule includes as part of its structure an activatable protein tyrosine kinase (PTK). Experiments were conducted to demonstrate that UV illumination activates the EGF-R PTK and that PD 153035 inhibits this activation; PD 153035 is a EGF-R inhibitor (commercially available from TOCRIS, Ballwin, Mo.), it is a brominated quinazoline developed by Parke-Davis (1994, Ann Arbor, Mich.). Cell cultures were tested either untreated or treated with one of EGF, IL-1, TNF, UV radiation, the treatment being performed either before or after pretreatment with PD 153035. After the treatment, cells extracts were subjected to immunoprecipitation with EGF-R antibody and then tested with an antibody to determine whether the tyrosine kinase part of EGF-R was activated. The receptor itself was tested for the EGF-R protein to assure it was, in fact, present (i.e., controls for the experiments which measured the total tyrosine kinase present, both phosphorylated and unphosphorylated). The results show a consistent and essentially constant amount of EGF-R protein, confirming that the receptor was present in all of the cell extracts. In comparison with untreated (UNTR) cells, EGF, UV, IL-1, and TNF were seen to activate EGF-R. However, when the cells were also treated with PD 153035 and the respective challenging agents, the amount of phosphorylated tyrosine kinase from EGF-R was essentially the same as that seen in untreated cells. Accordingly, PD 153035 clearly inhibits phosphorylation (activation) of the tyrosine kinase function of EGF-R.

MMPs may also be induced via IL-1, but because its receptor does not include protein tyrosine kinase activity as EGF-R does, it could be activated by recruiting a kinase. IRAK (IL-1 Receptor-Activated Kinase) is a protein tyrosine kinase (enzyme) that binds to and is activated by IL-1R (the IL-1 receptor) and in turn activates a pathway that leads to induction of c-JUN kinase, MMPs, and thus collagen degradation. Untreated cells in culture and cells in culture treated with PD 153035 had a minimal baseline amount of IRAK activity. In contrast, UV-irradiated, IL-1-treated, and EGF-treated cells were found to have a significant amount of IRAK acitivty in comparison with the baseline level. Cells treated with PD 153035 and then challenged with UV or EGF clearly had less phosphorylated IRAK than those without the PD 153035 pretreatment. However, PD 153035-treated cells exposed to IL-1 showed no reduction in phosphorylated IRAK. Thus, UV, IL-1, and EGF each induces IRAK phosphorylation, and pretreatment with PD 153035 inhibits the IRAK phosphorylation due to challenge with UV or EGF, but not when challenged with IL-1. These results are unexpected. While use of an EGF-R protein tyrosine kinase inhibitor might have been expected to inhibit the EGF-R activation by UV irradiation, it would not have been expected to inhibit the IL-1R activation by UV irradiation. While not desirous of being constrained to a particular theory of operation, it appears that there may be biochemical signalling (crosstalk) between the EGF-R pathway and the IL-1R pathway, where activation of the EGF-R pathway results in activation of the IL-1 pathway. Accordingly, if this finding is accurate, one can further explain our invention as the use of an EGF-R tyrosine kinase inhibitor to inhibit UV-induced MMPs from both pathways.

We also tested cultured human keratinocytes for c-JUN kinase activity after exposure to UV radiation, where some of the cells had been pretreated with PD 153035, a compound that specifically inhibits EGF-R. These cells were tested for phosphorylation of GST-c-jun (phospho-c-jun protein), which is catalyzed by c-JUN kinase. Untreated cells (UNTR) and cells not exposed to UV but treated with PD 153035 had a baseline amount of phospho-GST-c-jun protein. Cells exposed to UV radiation and not treated with PD 153035 showed a significant amount of phospho-GST-c-jun above the baseline amount. However, cells treated with PD 153035 and then exposed to UV radiation had phospho-GST-c-jun protein levels comparable with the baseline levels seen with unexposed cells (whether or not treated with PD 153035). These results show that PD 153035 inhibition of EGF-R inhibits UV activation of c-JUN kinase, which would otherwise lead to induction of MMPs and inhibition of collagen synthesis.

In addition to PD 153035, other classes of compounds are likely to be suitable, and especially those having a molecular weight of less than about 400 would likely be expected to be administrable transdermally via a cream, spray, or other suitable, cosmetically and dermatologically acceptable, formulation. Such compounds (as described in the aforementioned article by Noonberg and Benz) include genistein (4′,5,7-trihydroxyisoflavone), suramin sodium (and related derivatives), heribimycin-A, quercetin, lavendustin-A, erbstatin, benzylidenemalononitriles (referred to a tyrphostins, for tyrosine phosphorylation inhibitors), brominated quinazolines (such as PD-160678 and PD-168383), phenylamino- and pyrazolopyrimidine and pyrrolopyrimidine compounds (such as STI-571 and PKI-166), thioindoles, dianilinopthalimides, anthraquinones, and SU-5416 and SU-6668, and derivatives thereof. Using the techniques described herein, one can determine whether a given compound shows in vitro results.

Using the techniques described in the aformentioned U.S. Pat. Nos. 5,837,224 and 6,130,254, and the Ser. No. 28,435 application (the disclosures of which are all incorporated herein by reference), one can conduct in vivo experiments to determine actual efficacy of the compound on human skin.

Human volunteers, each having given informed consent, were used to determine the effect, if any, of pretreatment of their skin with an EGF-R PTK inhibitor prior to exposure of the skin to UV radiation. Hip or buttocks skin areas of the volunteers were pretreated using either our standard vehicle (70:30 of ethanol and propylene glycol), or a solution of 5% genistein (by weight) in DMSO. On the hip or buttock skin of volunteers, the test solution was placed (or on adjacent areas if both solutions were used), and the areas occluded for 24 hours; thereafter, the area was biopsied, or it was exposed to 2 MEDs of UV radiation and biopsied after exposure. The UV source was a bank of UVB fluorescent lamps model F36T12 (putting out 26% in visible and near IR wavelengths), filtered with Kodacel TA401/407 filter (available from Kodak, Rochester, N.Y.). Total irradiation 290-800 nm 17 inches from the source was 1.49×10−3 w/cm². Although the experiments were performed using a UVB source, to the extent that UVA radiation activates the EGF receptor, we would expect the results and treatment methods disclosed herein to function the same as with this UVB source.

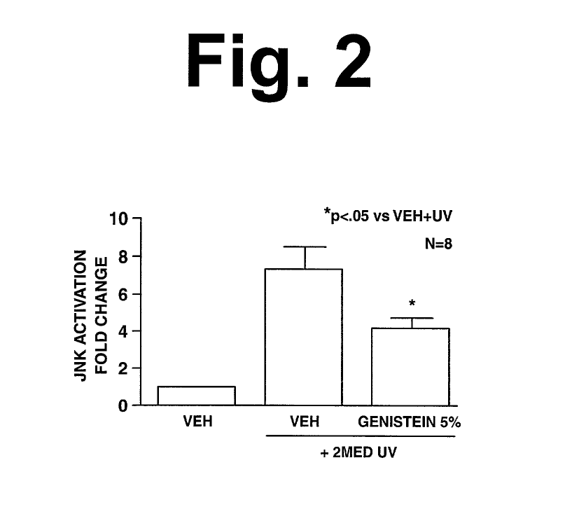

FIG. 2 depicts the results from the skin of volunteers tested for the change in JNK activation. As shown in FIG. 1, UV radiation and ROS activate the cytokine receptor pathway, which, through JNK, creates AP-1, leading to premature aging due to the sun. After the volunteers' skin was occuled for 24 hours, it was biopsied, and other areas were exposed to 2 MEDs of UV radiation and then biopsied about 4 hours thereafter. The results shown in FIG. 2 indicate that UV radiation significantly increased the activation of JNK, but that 5% genistein significantly reduced the amount of JNK activated. These results also indicate that the genistein solution was able to penetrate the skin. Thus, topical genistein is an effective composition for inhibiting photoaging through the cytokine pathway.

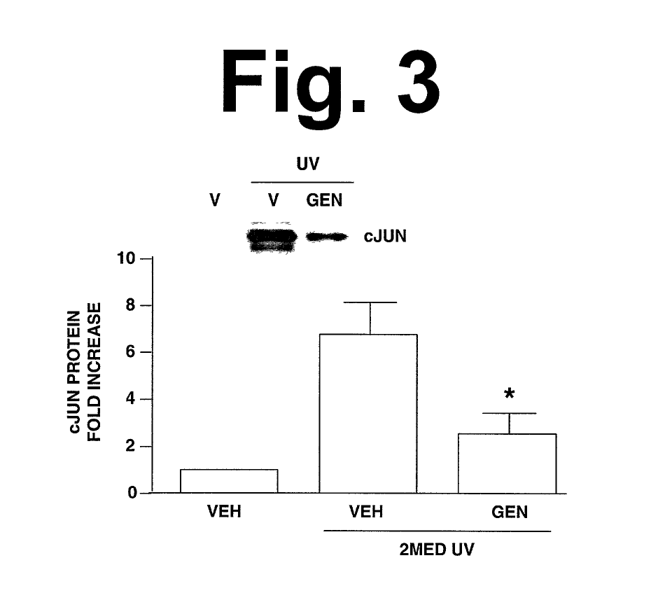

FIG. 3 depicts the results from the skin of volunteers tested for any changes in the amount of cJUN protein induced by UV radiation. The same procedure as described above was repeated, except that biopsy for cJUN protein was taken 8 hours after exposure to the UV radiation. As shown in the figure, topically applied genistein solution significantly inhibited the increased in the amount of cJUN protein in the skin after UV exposure, as compared with vehicle-treated skin. The inset in the figure is a Western blot showing the amount of cJUN protein in genistein-treated versus vehicle-treated skin.

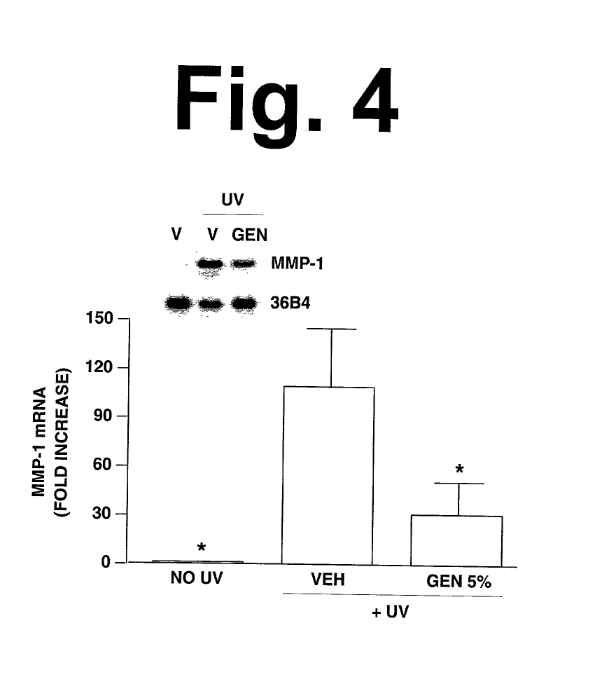

FIG. 4 depicts the results from the skin of volunteers tested for the change in the amount of MMP-1 mRNA induced by UV radiation. The same procedure as described above was repeated, except that biopsy for MMP-1 mRNA was taken 24 hours after exposure to the UV radiation. As shown in the figure, topically applied genistein solution significantly inhibited the increase in MMP-1 mRNA induced by the solar simulator in vehicle-treated skin. (The insert shows a Northern blot of the MMP-1 mRNA and that of the reporter gene 36B4.) Accordingly, topical administration of genistein has effects downstream, reducing the signalling that directly causes MMP-1 to be produced.

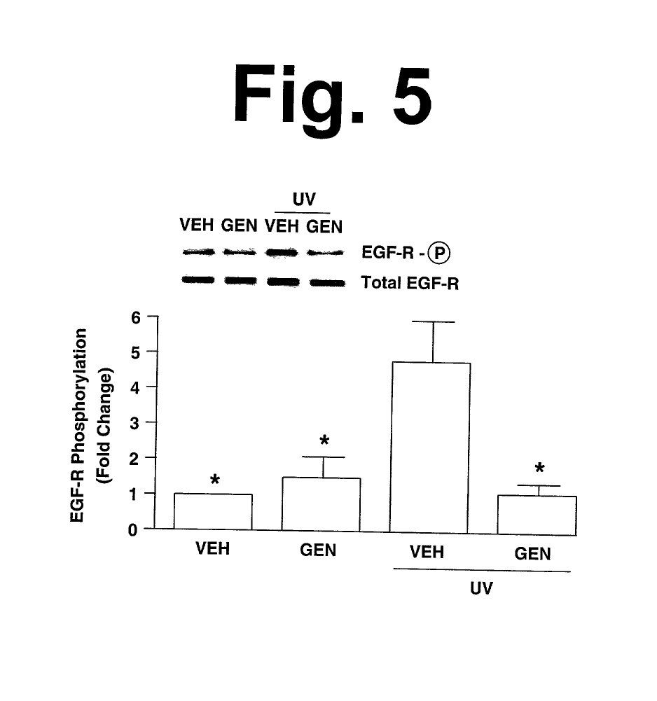

The just-described examples, the results of which are shown in FIGS. 2-4, evidence the ability of a compound like genistein to inhibit UV-induced cytokine signalling that results in up-regulation of MMPs. FIG. 5 depicts the results from the skin of volunteers tested for the amount of EGF-R phosphorylated after exposure to UV radiation. As described above, EGF-R is activated when phosphorylated. Reducing, if not preventing, phosphorylation of EGF-R would decrease its activity and the concomitant increase in MMPs after exposure to UV radiation. First, after the 24 hour occlusion, the volunteers' skin was biopsied tested to determine whether the vehicle alone or the genistein solution alone induced phosphorylation in EGF-R. The two left hand bars of the histogram in FIG. 5 indicate that the genistein solution did not induce EGF-R phosphorylation. As part of this same trial, the volunteers' skin was exposed to 2 MEDs of UV radiation, and thirty minutes (30 min.) after exposure their skin was again biopsied and tested. As shown by the right-hand portion of FIG. 5, genistein treated skin showed significantly less of the phosphorylation of EGF-R found in vehicle-treated skin. Accordingly, topically applied genistein inhibits the growth factor receptor pathway that leads to photoaged skin after exposure of the skin to UV radiation.

While EGF-R PTK inhibitors are believed to function much earlier in the pathways that lead to upregulation of MMPs and inhibition of collagen biosynthesis, there may also be some advantage to using these compounds in combination with retinoids and other MMP inhibitors, including direct acting MMP inhibitors, P-450 inhibitors (which inhibit the enzyme that degrades retinoic acid receptors in the skin), “antioxidants” (also appear to inhibit MMP upregulation), sunscreens, and the like; especially in that lower doses of compounds may likely be as efficacious when used in these types of combinations.

Genistein, and its β-glucoside conjugate genistin, can be found in soy milk, tofu (bean curd), miso (bean paste), natto (fermented soybeans), and soy sauce. Other natural EGFR activation inhibitors, and derivatives thereof, include staurosporine, aeroplysinin (K. Hinterding et al., “Synthesis and biological evaluation of aeroplysinin analogues: a new class of receptor tyrosine kinase inhibitors,” Bioorg Med Chem 1998 Aug; 6(8):1153-62; H. Waldmann et al., “Selective Inhibition of Receptor Tyrosine Kinases by Synthetic Analogues of Aeroplysinin,” Angew. Chem. Int. Ed. Engl. 1997, 36, No. 13-14, 1541-1542), lavendustin A (M. S. Symth et al., “Non-amine based analogues of lavendustin A as protein-tyrosine kinase inhibitors,” J Med Chem 1993 Oct 1; 36(20):3010-4), piceatannol (3,4,3′,5′-tetrahydroxy trans stilbene, a plant secondary natural product; N. C. Mishra et al., “Inhibitory effect of piceatannol, a protein tyrosine kinase inhibitor, on asexual maturation of Plasmodium falciparum,” Indian J Exp Biol 1999 Apr; 37(4):418-20; K. Thakkar, “Synthesis and protein-tyrosine kinase inhibitory activity of polyhydroxylated stilbene analogues of piceatannol,” J Med Chem 1993 Oct 1; 36(20):2950-5), hymenialdisine (SK&F 108752) and herbimycin (A. M. Badger et al., “Inhibition of interleukin-1-induced proteoglycan degradation and nitric oxide production in bovine articular cartilage/chondrocyte cultures by the natural product, hymenialdisine,” J Pharmacol Exp Ther 1999 Aug; 290(2):587-93), kaempferol and quercetin (and the kaempferol glycosides kaempferol-O-3-alpharhamnopyranoside and kaempferol-O3-alpha-arabinopyranoside, M. Abou-Shoer et al., “Flavonoids from Koelreuteria henryi and other sources as protein-tyrosine kinase inhibitors,” J Nat Prod 1993 Jun; 56(6):967-9; M. Cushman et al., “Synthesis and protein-tyrosine kinase inhibitory activities of flavonoid analogues,” J Med Chem 1991 Feb; 34(2):798-806), and erbstatin and tyrphostins (e.g., M. Treuner et al., “Limited selectivity of a synthetic erbstatin derivative for tyrosine kinase and cell growth inhibition,” Biochem Int 1992 Mar; 26(4):617-25).

One screening method for determining the ability of a given compound to inhibit the activation of EGFR is to use cultured cells or an organ culture, preferably using human cells (such as the human skin organ culture described by S. W. Stoll and J. T. Elder, “Retinoid regulation of heparin-binding EGF-like growth factor gene expression in human keratinocytes and skin”, Exp. Dermatol., 1998: 7: 391-397) that have been challenged with an agonist known to induce EGFR activation, such as EGF. Although not essential, but desirable, the test agonist compound can also be used in combination with a Western blot to assure that the total amount of EGFR is unchanged and that only the amount of EGFR activated/phosphorylated is increased (as was the case with the experiments shown in FIG. 5). The cultured cells or organ culture are exposed to the desired agonist compound, then the test inhibitor compound is added, and finally the cells are examined (such via Western blot) to determine the extent of EGFR activation.

The amount of inhibitor used therapeutically depends on the selectivity of the inhibitor for the EGFR, whether it is a reversible or irreversible inhibitor, its ability to penetrate the skin (the composition may include a penetration enhancer), its stability, its metabolism, and the like. In general, 0.1% to 10%, more preferably about 5% by weight of the composition of a reversible inhibitor is used; lesser amounts of an irreversible inhibitor are used. A combination of reversible and irreversible inhibitors can also be used.

Retinoids include natural and synthetic analogs of vitamin A (retinol), vitamin A aldehyde (retinal), vitamin A acid (retinoic acid (RA)), including all-trans, 9-cis, and 13-cis retinoic acid), etretinate, and others as described in EP-A2-0 379367, U.S. Pat. Nos. 4,887,805, and 4,888,342 (the disclosures of which are all incorporated herein by reference). Various synthetic retinoids and compounds having retinoid activity are expected to be useful in this invention, to the extent that they exhibit retinoid activity in vivo, and such are described in various patents assigned on their face to Allergan Inc., such as in the following U.S. Pat. Nos. 5,514,825; 5,698,700; 5,696,162; 5,688,957; 5,677,451; 5,677,323; 5,677,320; 5,675,033; 5,675,024; 5,672,710; 5,688,175; 5,663,367; 5,663,357; 5,663,347; 5,648,514; 5,648,503; 5,618,943; 5,618,931; 5,618,836; 5,605,915; 5,602,130. Still other compounds described as having retinoid activity are described in other U.S. Pat. Nos. 5,648,563; 5,648,385; 5,618,839; 5,559,248; 5,616,712; 5,616,597; 5,602,135; 5,599,819; 5,556,996; 5,534,516; 5,516,904; 5,498,755; 5,470,999; 5,468,879; 5,455,265; 5,451,605; 5,343,173; 5,426,118; 5,414,007; 5,407,937; 5,399,586; 5,399,561; 5,391,753; and the like, the disclosures of all of which are incorporated herein by reference.

MMPs are also inhibited by BB2284 (described by Gearing, A. J. H. et al., Nature (1994) 370:555-557), GI129471 (described by McGeehan G. M., et al., Nature (1994) 370:558-561), and TIMPs (tissue inhibitors of metalloproteinases, which inhibit vertebrate collagenases and other metalloproteases, including gelatinase and stromelysin). Still other compounds useful for the present invention include direct inhibitors of MMPs, such as hydroxamate and hydroxy-urea derivatives, including those such as Galardin, Batimastat, and Marimastat, and those disclosed in EP-A1-0 558635 and EP-A1-0 558648 (as useful for inhibiting MMPs in the treatment of, among other etiologies, skin ulcers, skin cancer, and epidermolysis bullosa). Retinoids have been reported by Goldsmith, L. A. (Physiology, Biochemistry, and Molecular Biology of the Skin, 2nd. Ed. (New York: Oxford Univ. Press, 1991), Chpt. 17) to cause an increase in steady state levels of TIMP mRNA that would suggest transcriptional control; although, based on our discoveries, we have found this is not true in human skin in vivo.

Any drug which inhibits the cytochrome P-450 enzymes that metabolize retinoic acid can also be useful in practicing this invention. In the skin, retinoids are converted into retinoic acide (RA) as the active form. Retinoic acid (RA) is then metabolized to inactivation by hydroxylation (via RA 4-hydroxylase) to 4-hydroxy-RA, which is then oxidized to 4-oxo-RA by a reaction mediated by a cytochrome P-450-dependent monooxygenase system. (S. Kang et al., “Liarozole Inhibits Human Epidermal Retinoic Acid 4-Hydroxylase Activity and Differentially Augments Human Skin Responses to Retinoic Acid and Retinol In Vivo,” J. Invest. Dermatol., 107:183-187 (Aug. 1996); E. A. Duell et al., “Human Skin Levels of Retinoic Acid and Cytochrome P-450-derived 4-Hydroxyretinoic Acid after Topical Application of Retinoic Acid In Vivo Compared to Concentrations Required to Stimulate Retinoic Acid Receptor-mediated Transcription In Vitro,” J. Clin. Invest., Skin Retinoid Levels and Reporter Gene Activity, 90:1269-1274 (Oct. 1992); E. A. Deull et al., “Retinoic Acid Isomers Applied to Human Skin in Vivo Each Induce a 4-Hydroxylase That Inactivates Only Trans Retinoic Acid,” J. Invest. Dermatol., 106:316-320 (Feb. 1996); the disclosures of which are incorporated herein by reference). Accordingly, compounds which interfere with the elimination metabolism of all trans RA, the active metabolite of topically applied retinoids such as 9-cis RA and 13-cis RA, will beneficially increase the amount of RA in the skin. Thus, preventing the degradation of natural (all trans) RA in the skin effectively increases its concentration, and so provides the benefits described herein. Examples of compounds dermatologically acceptable and having or likely to have inhibitory effects on the P-450-mediated degradation of RA include azoles, especially triazoles, including, for example, ketoconazole (U.S. Pat. Nos. 4,144,346 and 4,223,036), fluconazole (U.S. Pat. No. 4,404,216), itraconazole (U.S. Pat. No. 4,267,179), liarozole, irtemazole, and the like; compounds related to these that may also be useful include, for example, diazines such as flucytosine. It would also be beneficial to use such cytochrome P-450 inhibitors in combination with a reduced amount of retinoid; the P-450 inhibitor decreases the metabolic elimination of the retinoid and so less retinoid is needed to achieve the same result. Still further, analytical methods are available for determining whether a given compound inhibits the degradation of RA by applying the compound and testing for changes in CRABP (cytoplasmic retinoic acid binding protein), which will have increased levels if the levels of RA are also increased by the topical application of the test compound.

Still other inhibitors of MMPs that can be applied topically and are useful in practicing the claimed invention include the tetracyclines and derivatives thereof, such as minocycline, roliteracycline, chlortetracycline, methacycline, oxytetracycline, doxycycline, demeclocycline, and the various salts thereof. Because of possible allergic or sensitization reactions, the topical adminstration of tetracyclines should be monitored carefully for such untoward reactions.

MMP inhibitors also include genistein and quercetin (as described in U.S. Pat. Nos. 5,637,703, 5,665,367, and FR-A-2,671,724, the disclosures of which are incorporated herein by reference) and related compounds, as well as other antioxidants such as NAC (N-acetyl cystein), green tea extract, and others. Although NAC is the precursor to the powerful antioxidant glutathione, human skin is significantly more permeable to NAC than to glutathione, and so it is more suitable for the topically applied compositions. Antioxidants also can be viewed as MMP inhibitors to the extent that they might function by quenching or otherwise reducing free radicals and reactive oxygen species which initiate or lead to MMP induction, such as via the MAP kinase cascade. Antioxidants include glutathione and its precursors, such as N-acetyl cysteine (NAC) (as mentioned above), more broadly N—CH₃(CH₂)ₙCO cysteine (wherein n is an integer from zero to eight, more preferably not more than 4), and related compounds and derivates thereof as described in U.S. Pat. No. 5,296,500 (the disclosure of which is incorporated herein by reference). Antioxidants also include: (i) lipid-soluble compounds such as β-carotene and its derivatives, other carotenoids, and vitamin E and related tocopherols; (ii) water-soluble compounds such as vitamin C, glutathione, and NAC; and (iii) other compounds (such as one of the pigments that makes tomatoes red, and lipoic acid found in potatoes).

Various UV blockers are known in the paint and dye industry to prevent pigment or color degradation of cars, homes, and clothing. A particularly preferred UVA_{½}-blocker for use on human skin is PARSOL® 1789 and PARSOL® MCX (Schering-Plough), as well as those mentioned in U.S. Pat. No. 4,387,089, which describes the preparation of this UVA-blocker. We have found that true UVA blockers inhibit induction of cJUN mRNA and of collagenase and gelatinase. Most preferably, UV blockers should block radiation of both less than about 320 nm and between about 380 and 390 nm. Other sunscreen compositions are described in our co-pending application No. 60/216244, filed Jul. 6, 2000, and the above-mentioned U.S. Pat. No. 6,130,254, the disclosures of which are incorporated herein by reference.

Various changes, modification, and additions may become apparent to one of ordinary skill in these arts, and such within the spirit of this invention are intended to be included with the scope of the claims appended hereto.

What is claimed is:

1. A method for preventing photoaging in human skin, by administering an EGF-R protein tyrosine kinase inhibitor to the human whose skin is exposed to UV radiation.

2. The method of claim 1, wherein the administration is topical.

3. The method of claim 1, wherein the administration is prior to exposure to UV radiation.

4. The method of claim 3, wherein the administration is at least six (6) hours prior to exposure.

5. The method of claim 1, wherein the tyrosine kinase inhibitor is selected from the group consisting of isoflavones, suramin sodium (and related derivatives), heribimycin-A, lavendustin-A, erbstatin, benzylidenemalononitriles, brominated quinazolines, tyrphostins, phenylaminopyridines, pyrazolopyrimidines, pyrrolopyrimidines, thioindoles, dianilinopthalimides, anthraquinones, and mixutres thereof.

6. The method of claim 4, further comprising administering a retinoid.

7. The method of claim 5, wherein the isoflavone is genistein or quercetin.

8. A composition for preventing induction of MMPs in human skin due to exposure of the skin to UV radiation, comprising an EGF-R protein tyrosine kinase inhibitor admixed in a dermatologically suitable carrier therefor.

9. The composition of claim 8, further comprising at least one additional compound selected from the group consisting of retinoids, P-450 inhibitors, antioxidants, UV sunscreens, and compatible mixtures thereof.

10. The composition of claim 9, comprising a UVA blocker and a UVB blocker, and at least one additional compound selected from the group consisting of retinoids, P-450 inhibitors, and antioxidants, and compatible mixtures thereof.

11. The composition of claim 10, wherein the additional compound is a retinoid.

12. The composition of claim 11, wherein the retinoid is retinol.

13. A composition for preventing induction of MMPs in human skin due to exposure of the skin to UV radiation, comprising an EGF-R protein tyrosine kinase inhibitor and a retinoid admixed in a dermatologically suitable carrier therefor.

14. The composition of claim 13, wherein the retinoid is retinol or retinoic acid.

15. The composition of claim 13, wherein the EGF-R inhibitor is an isoflavone.

16. The composition of claim 15, wherein the isoflavone is genistein.
